(19) United States
(12) Patent Application Publication
(10) Pub. No.: US 2007/0144550 A1
(43) Pub. Date: Jun. 28, 2007

(54) Device and system for applying a hair coloring product to a lock of hair

(76) Inventor: Kathy Roher, Hartsdale, NY (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/318,462

(22) Filed: Dec. 28, 2005

(51) Int. Cl. A61K 8/18

(52) U.S. Cl. 132/208

(57) ABSTRACT

A device for applying a hair coloring product to a lock of scalp hair includes a pair of gripping members. Each gripping member comprises a free end including an inner faces, an outer face, and a plurality of fibers extending from the inner face. The pluralities of fibers are configured to be loaded with the hair coloring product, and the device is configured so that application of a pressing force to at least one of the gripping members moves at least one of the inner faces toward the other inner face so as to enable application of the hair coloring product to a lock of hair positioned between the inner faces.

### BACKGROUND

1. Field of the Invention

The present invention relates to coloring of scalp hair. More particularly, the present invention relates to a device and system for the application of a coloring product (e.g., a hair highlighting product) to a lock of human scalp hair.

2. Description of the Related Art

Hair coloration is a very popular way of changing an individual's appearance. Techniques and products for coloring hair span a vast array of different approaches. Some hair coloring processes are typically practiced only by professionals in salons. Other, more recent approaches, involve hair coloring products sold in retail stores that permit individuals to color their own hair without the need to visit a salon.

One very common technique of hair coloring, referred to as “highlighting,” involves selecting particular strands of hair to be colored—a “lock” of hair—and applying a coloring product to the hair lock. When multiple locks of hair are colored, the overall effect sometimes creates an appearance of hair that is layered and more full. Some highlighting techniques involve a hair coloring product being applied to an entire lock of hair, extending from the roots to the tips, while other techniques involve product being applied only in the tip regions.

In one prior approach typically used by salon professionals for coloring locks of hair, a hair coloring product is brushed onto a lock of hair and then a protective film is wrapped around the colored lock, thereby preventing the product from contacting other hair not intended to be colored. The procedure is repeated for each lock of hair to be treated. This method is cumbersome in that it requires an adequate view of the entire scalp and also presents significant maneuverability issues for the user, particularly the self-applying user. Further, failure to execute the tasks with adequate precision may result in undesirable effects, for example, uneven color application and extraneous hair coloration.

In the current state of the art, there are many devices intended to simplify the application of hair coloring products, but a number of those devices have certain drawbacks and disadvantages. For example, U.S. Pat. No. 6,250,312 teaches an apparatus comprised of a notched brush and a molded hair guide affixed to each end of a tong-like handle. Such a device may be problematic in that it may cause individual hairs within a strand to become gripped and/or tangled in brush bristles, the molded guide, and/or the handles of the device, thereby pulling hair from the head painfully as the device is moved from the scalp to the tip end of the lock.

U.S. Pat. No. 5,676,480 describes an apparatus with semi-rigid applicator members formed of sponge material and spaced apart sufficiently to receive a small grouping of hairs therebetween. Such an arrangement while enabling one with ample dexterity and range of motion to carefully align a “small” grouping of hair between its members, is unable to accommodate larger locks of hair and also poses difficulties for a less dexterous user in aligning a desired lock of hair between the applicator members. Furthermore, it may be difficult for a user to grip a lock of hair between the applicator members so as to provide sufficient application of a hair coloring product. These difficulties may become particularly evident while attempting to self-apply a hair coloring product to locks of hair which are on the back side of the scalp (e.g., those requiring an operator to work while looking in the mirror).

Although the present invention may obviate one or more of the above-mentioned drawbacks, it should be understood that some aspects of the invention might not necessarily obviate one or more of those drawbacks.

### SUMMARY

In the following description, certain aspects and embodiments of the present invention will become evident. It should be understood that the invention, in its broadest sense, could be practiced without having one or more features of these aspects and embodiments. In other words, these aspects and embodiments are merely exemplary.

One aspect of the invention relates to a system for coloring a lock of scalp hair, including a hair coloring product and a device for applying the product. The hair coloring product may be a product to be applied to the lock of scalp hair. The device may include a first gripping member comprising a first end portion having a free end and including a first inner face and a first outer face, and a first plurality of fibers extending from the first inner face, and a second gripping member comprising a second end portion having a free end and including a second inner face and a second outer face, a second plurality of fibers extending from the second inner face. The first and second pluralities of fibers may be configured to be loaded with the hair coloring product. The device may be configured so that application of a pressing force to at least one of the first and second gripping members moves at least one of the first and second inner faces toward the other inner face so as to enable application of the hair coloring product to a lock of hair between the first and second inner faces.

The term “hair coloring product,” as used herein, should be understood to mean any type of coloring product (including, e.g., a product chosen from dyes, highlights, bleaches, and/or any other substances for altering the color of hair) specifically meant for application to hair growing from or otherwise affixed to a human scalp. Therefore, the term hair coloring product specifically excludes mascara and other products meant for specific application only to eyelashes, eyebrows, and/or hair other than that growing from or otherwise affixed to a human scalp.

Another aspect of the invention includes a device for applying a hair coloring product to a lock of scalp hair. The device may include a first gripping member comprising a first end portion having a free end and including a first inner face and a first outer face, and a first plurality of fibers extending from the first inner face, wherein the first outer face is substantially free of fibers. The device may also include a second gripping member comprising a second end portion having a free end and including a second inner face and a second outer face, and a second plurality of fibers extending from the second inner face, wherein the second outer face is substantially free of fibers. At least a portion of the first gripping member opposite the first end portion and at least a portion of the second gripping member opposite the second end portion may be defined by a unitary, single piece of a plastic material. In an at rest position, each of the first inner face and the second inner face may extend in a respective direction that slopes away from the other inner face such that spacing between the first and second inner faces increases closer to the free ends of the first and second end portions. The first and second pluralities of fibers may be configured to be loaded with the hair coloring product. The device may be configured so that application of a pressing force to at least one of the first and second gripping members moves at least one of the first and second inner faces toward the other inner face so as to enable application of the hair coloring product to a lock of hair between the first and second inner faces.

Aside from the structural and procedural arrangements set forth above, the invention could include a number of other arrangements such as those explained hereinafter. It is to be understood that both the foregoing description and the following description are exemplary only.

### BRIEF DESCRIPTION OF THE DRAWINGS

The accompanying drawings are incorporated in and constitute a part of this specification. The drawings illustrate at least one exemplary embodiment and, together with the description, serve to explain some principles of the invention. In the drawings,

FIG. 1 shows a side view of device for applying a hair coloring product consistent with an embodiment of the present invention;

FIG. 2 shows a perspective view of the device of FIG. 1;

FIG. 3 is an exemplary depiction of the device of FIG. 1 being used for applying a hair coloring product to a lock of hair; and

FIG. 4 is a partial, exploded view of an exemplary system including the device of FIG. 1.

### DESCRIPTION OF EXEMPLARY EMBODIMENTS

Reference will now be made in detail to exemplary embodiments of the invention, an example of which is illustrated in the accompanying drawings. Wherever possible, the same reference numbers will be used throughout the drawings to refer to the same or like parts.

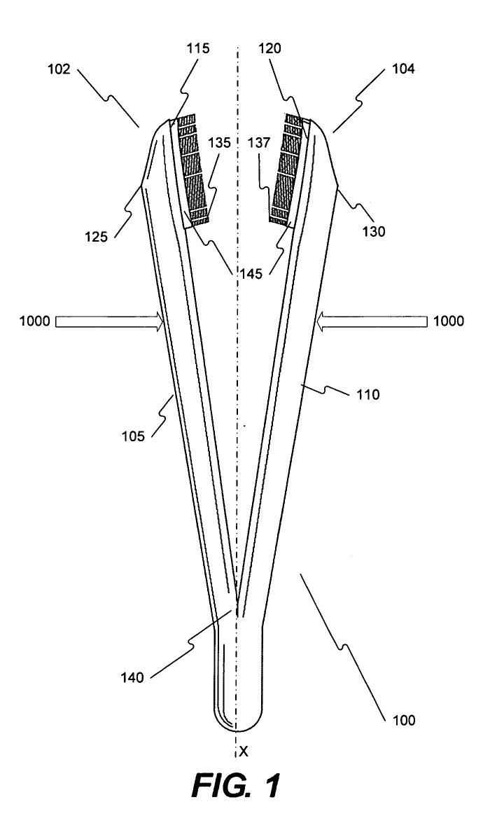

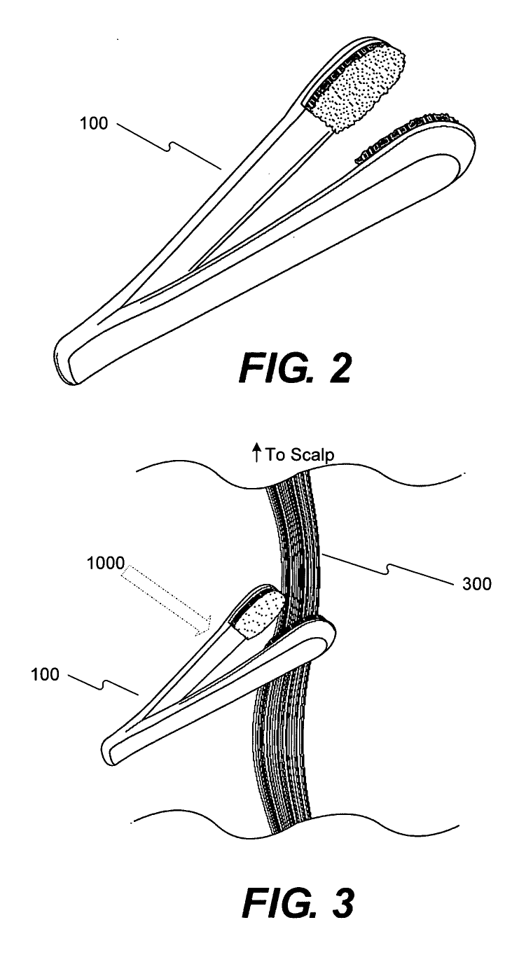

FIGS. 1-3 show a device 100 for applying a hair coloring product consistent with an embodiment of the present invention. As shown in FIG. 1, device 100 includes a first gripping member 105 and a second gripping member 110. First gripping member 105 and second gripping member 110 may be arranged in many different ways and fabricated from any suitable material. In the example of FIG. 1, gripping members 105 and 110 are defined by a single piece of plastic material, for example, polyethylene or polypropylene, such that first gripping member 105 and second gripping member 110 originate from a common point of “connection” at articulation 140. When device 100 is in an at rest position (e.g., when a pressing force 1000, described below, is not present), each gripping member slopes in a respective direction away from the X-axis so as to form a V-shaped, opposable arrangement between first gripping member 105 and second gripping member 110 (either substantially symmetric about axis x or non-symmetric). Gripping members 105 and 110 have a first free end 102 and a second free end 104, respectively.

In some alternative arrangements (not shown), first gripping member 105 and second gripping member 110 may be defined by respective, individual pieces of material and connected together at articulation 140 through a connection other than a single piece of material, using, for example, an adhesive or a hinge/spring.

Although many variances are possible, in the embodiment of FIG. 1, the length of first gripping member 105 and the length of second gripping member 110 may be substantially equal. Further, the lengths of gripping members 105 and 110 may be sufficient such that a separation distance between free ends 102 and 104 is relatively large enough to accommodate a relatively large lock of hair while not causing an uncomfortable grip in an average-sized human hand.

Pressing force 1000 applied to one or both of the outer faces of the gripping members 105 and 110 causes an inner face of one or more of the gripping members to be moved closer to the other face. First gripping member 105 and second gripping member 110 may have a thickness sufficient so as to maintain adequate flexibility near articulation 140 while also being formed of a material having sufficient resiliency to return device 100 to its at-rest position following release of pressing force 1000.

As shown in FIG. 1, a portion of first member 105 including first free end 102 and a portion of second member 110 including second free end 104 are substantially similar to one another. In particular, the members 105 and 110 have inner faces 115 and 120, outer faces 125 and 130, and pluralities of fibers 135 and 137 respectively. Inner faces 115 and 120 may be substantially planar. In addition (or alternatively) inner faces 115 and 120 may have a slight concavity. When device 100 is at rest, the distance between first inner face 115 and second inner face 120 increases in a direction along the X-axis away from articulation 140 and toward free ends 102 and 104. Alternatively, the distance between first inner face 115 and second inner face 120 may remain equal or may decrease in a direction along the X-axis and away from articulation 140 and toward free ends 102 and 104. In the preferred embodiment, application of pressing force 1000 to one or both gripping members may cause first inner face 115 and second inner face 120 to move toward one another enabling a lock of hair to be contacted by first plurality of fibers 135 and second plurality of fibers 137.

First plurality of fibers 135 and second plurality of fibers 137 extend away from inner faces 115 and 120. The fibers 135 and 137 have a length and flexibility such that the fibers 135 and 137 have substantial pliability. First plurality of fibers 135 and second plurality of fibers 137 are configured to be loaded with a hair coloring product which may be applied to a lock of hair by positioning the lock between the members, as shown in FIG. 3, applying the pressing force 1000 to one or both members 105 and 110, and then moving the device along the length of the lock. Like materials may be used for forming both first plurality of fibers 135 and second plurality of fibers 137. Alternatively, first plurality of fibers 135 and second plurality of fibers 137 may be formed of dissimilar materials in order to achieve particular effects or results during the coloring process.

The pluralities of fibers may be fabricated from any material generally suitable for being loaded with and applying hair coloring products. For example, fiber materials may include polypropylene, nylon, mohair, or any combination of other suitable materials consistent with the present invention. In some examples, the fibers may be in the form of flockings.

The fibers may be affixed to backing material 145 or may be affixed directly to inner faces 115 and 120. For example, the fibers may be woven into a fabric or mesh backing or may be affixed to backing material 145 using an adhesive or other technique. Further, where fibers are affixed to backing material 145, backing material may then be affixed to inner faces 115 and 120 using any suitable adhesive.

As noted, backing material 145 may be a fabric or mesh like material. Alternatively (or additionally), backing material 145 may comprise a sponge-like material or a materials similar to blown rubber (e.g., small-cell neoprene).

For each of the pluralities of fibers 135 and 137, the fibers may be grouped into tufts, each having a series of individual fibers bundled together so that fibers in the same tuft are spaced close to one another adjacent to inner faces 115 and 120 while diverging from each other at their free ends. At least some of the tufts may be spaced from one another and generally aligned so as to form a series of rows on backing material 145 or inner faces 115 and 120.

In some examples such as the embodiment of FIG. 1, the fibers 135 and 137 (and possibly also the optional backing material 145) may be defined by material typically used for painting pads for applying paint to walls.

First outer face 125 and second outer face 130 may be generally free of any fibers. The shape of first outer face 125 and second outer face 130 may be constructed so as to allow for comfortable gripping of device 100 and application of pressing force 1000 by fingers on an average-sized hand.

FIG. 3 is an exemplary depiction of the device being used for applying a hair coloring product to a lock of hair. Depending on the particular hair product, a user of device 100 may prepare the product in accordance with instructions associated with the product. Preparation may include, for example, opening one or more containers, mixing one or more liquids and/or powders, pouring the product, and/or any other action for making the hair coloring product usable for purposes described herein.

Once a hair coloring product has been prepared, first plurality of fibers 135 and second plurality of fibers 137 may then be loaded with the hair coloring product. Loading of the hair coloring product may be accomplished by, for example, dipping the fibers into the product, spraying the product onto the fibers, otherwise coating the fibers with product, or any other method known in the art for dispensing hair coloring product onto an applicator. Alternatively, only one of the first or second plurality of fibers may be loaded with the hair coloring product and the other plurality of fibers may be loaded with a different product or possibly even not loaded at all. When loaded with the hair coloring product, the plurality of fibers may become completely saturated, or alternatively, loading may be performed so as to only lightly load the fibers with hair coloring product.

A lock of hair 300 intended for coloring may be selected and isolated by the user at any time during the hair coloring process. For example, isolation of lock of hair 300 may occur after the hair coloring product has been prepared and after loading of the fibers with the hair coloring product has occurred. Once lock of hair 300 has been isolated, device 100 may be aligned, at a desired starting point for coloration (e.g., typically near the scalp), such that lock of hair 300 is located between first inner face 115 and second inner face 120 of device 100. The user may grip first gripping member 105 and second gripping member 110 while maneuvering device 100 for alignment. When lock of hair 300 is positioned as desired, a user may apply pressing force 1000, thereby causing first plurality of fibers 135 and second plurality of fibers 137 to contact lock of hair 300. Such contact may cause the hair coloring product present on the fibers to be dispensed upon lock of hair 300. The user may then move device 100, while in its gripping position (i.e., with continuous pressing force 1000), along lock of hair 300 in a direction away from the scalp and toward the tip of lock of hair 300, thereby substantially saturating lock of hair 300 with the hair coloring product. Due to the flexibility of the fibers, the hair coloring product may be applied without applying a substantial pulling force to the hair of the lock.

The user may repeat the above steps as desired to produce the desired hair coloring effect.

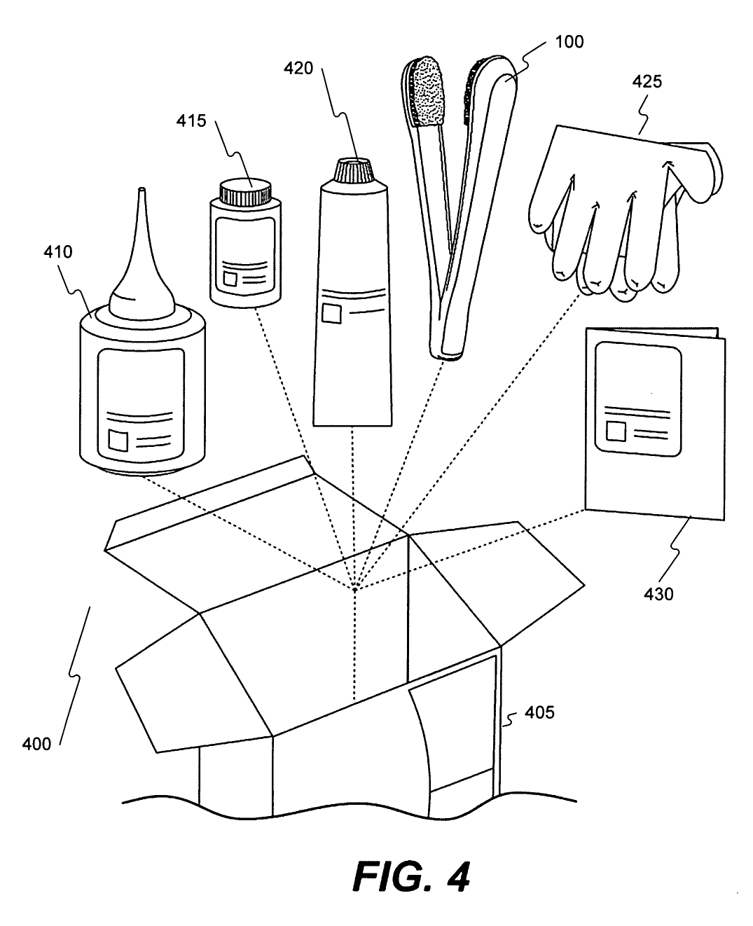

FIG. 4 is an exemplary depiction of a system for coloring hair consistent with an embodiment of the present invention. Hair coloring system 400 may include, for example, the device 100, one or more containers 410 and 415 containing a hair coloring product (e.g., a dye and developer and/or highlighting substance), a tube containing a conditioner 420, protective gloves 425, and printed instruction 430, which all may be contained in a packaging 405. Hair coloring system 400 may contain more or fewer components as desired.

Packaging 405 may be, for example, a box, a tin, a blister package, or any other package which may be used for containing the contents of the hair coloring system. Further, packaging 405 may also display instructions for use.

The hair coloring product of the system may be any hair coloring product designed to be applied to hair growing from or otherwise affixed to a scalp. Such a product may be used for lightening, bleaching, darkening, or otherwise changing the color of a lock of hair.

The conditioner may be used to condition hair following the application of the hair coloring product. For example, the conditioner may be a “super-conditioning” product or a standard conditioner designed to minimize damage to colored hair.

Protective gloves 425 may be provided in order to protect the user's hands from coming into direct contact with hair coloring product.

Instructions 430 may include instructions for preparing hair coloring product and using device 100. Instructions 430 may also depict and/or describe a step-by-step process for utilizing the system for coloring hair.

It will be apparent to those skilled in the art that various modifications and variations can be made to the structures and methodologies described herein. Thus, it should be understood that the invention is not limited to the subject matter discussed in the specification. Rather, the present invention is intended to cover modifications and variations.

I claim:

1. A system for coloring a lock of scalp hair, comprising:
a hair coloring product, the hair coloring product being a product to be applied to the lock of scalp hair; and
a device for applying the hair coloring product to the lock of scalp hair, the device comprising
a first gripping member comprising
a first end portion having a free end and including a first inner face and a first outer face, and
a first plurality of fibers extending from the first inner face, and
a second gripping member comprising
a second end portion having a free end and including a second inner face and a second outer face,
a second plurality of fibers extending from the second inner face,
wherein the first and second pluralities of fibers are configured to be loaded with the hair coloring product, and
wherein the device is configured so that application of a pressing force to at least one of the first and second gripping members moves at least one of the first and second inner faces toward the other inner face so as to enable application of the hair coloring product to a lock of hair between the first and second inner faces.

2. The system of claim 1, wherein the hair coloring product is a hair coloring product that highlights the lock of hair.

3. The system of claim 1, wherein at least some of the first and second pluralities of fibers comprise tufts of fibers.

4. The system of claim 1, wherein an end portion of the first gripping member opposite the first end portion and an end portion of the second gripping member opposite the second end portion are connected to one another, and wherein at least one of the first and second gripping members is sufficiently resilient so as to cause the device to at least substantially return to an at-rest position when the pressing force is removed.

5. The system of claim 4, wherein the first gripping member and the second gripping member are configured to form a substantially V shape when the device is in the at-rest position.

6. The system of claim 5, wherein the first inner face and the second inner face are not parallel to one another when the device is in the at-rest position.

7. The system of claim 1, wherein at least one of the first inner faces is substantially planar.

8. The system of claim 1, wherein the first plurality of fibers and the second plurality of fibers are substantially pliable.

9. A method of applying a hair coloring product to a lock of scalp hair, the method comprising:
loading at least one of the first and second pluralities of fibers of the system of claim 1, with the hair coloring product;
applying the pressing force to move at least one of the first and second inner faces toward the other inner face while the lock of hair is between the first and second inner faces; and
applying the hair coloring product to the lock of hair.

10. A device for applying a hair coloring product to a lock of scalp hair, comprising:
a first gripping member comprising
a first end portion having a free end and including a first inner face and a first outer face, and
a first plurality of fibers extending from the first inner face,
wherein the first outer face is substantially free of fibers; and
a second gripping member comprising
a second end portion having a free end and including a second inner face and a second outer face, and
a second plurality of fibers extending from the second inner face,
wherein the second outer face is substantially free of fibers,
wherein at least a portion of the first gripping member opposite the first end portion and at least a portion of the second gripping member opposite the second end portion are defined by a unitary, single piece of a plastic material,
wherein when the device is in an at rest position, each of the first inner face and the second inner face extends in a respective direction that slopes away from the other inner face such that spacing between the first and second inner faces increases closer to the free ends of the first and second end portions, and
wherein the first and second pluralities of fibers are configured to be loaded with the hair coloring product, and
wherein the device is configured so that application of a pressing force to at least one of the first and second gripping members moves at least one of the first and second inner faces toward the other inner face so as to enable application of the hair coloring product to a lock of hair between the first and second inner faces.

11. A system comprising:
a hair coloring product, the hair coloring product being a product to be applied to a lock of scalp hair; and
the device of claim 10.

12. The system of claim 11, wherein the hair coloring product is a hair coloring product that highlights the lock of hair.

13. The device of claim 10, wherein at least some of the first and second pluralities of fibers comprise tufts of fibers.

14. The device of claim 10, wherein the first gripping member and the second gripping member are configured to form a substantially V shape when the device is in the at-rest position.

15. The device of claim 10, wherein the first inner face and the second inner face are not parallel to one another when the device is in the at-rest position.

16. The device of claim 10, wherein at least one of the first inner faces is substantially planar.

17. The device of claim 10, wherein the first plurality of fibers and the second plurality of fibers are substantially pliable.

18. A method of applying a hair coloring product to a lock of scalp hair, the method comprising:
loading at least one of the first and second pluralities of fibers of the device of claim 10, with the hair coloring product;
applying the pressing force to move at least one of the first and second inner faces toward the other inner face while the lock of hair is between the first and second inner faces; and applying the hair coloring product to the lock of hair.
